(19) United States
(12) Patent Application Publication
(10) Pub. No.: US 2019/0015052 A1
(43) Pub. Date: Jan. 17, 2019

(54) MACHINE-LEARNING-BASED DENOISING OF DOPPLER ULTRASOUND BLOOD FLOW AND INTRACRANIAL PRESSURE SIGNAL

(71) Applicant: THE REGENTS OF THE UNIVERSITY OF CALIFORNIA, Oakland, CA (US)

(72) Inventors: Fabien Scalzo, Tarzana, CA (US); Abhejit Rajagopal, Irvine, CA (US)

(73) Assignee: THE REGENTS OF THE UNIVERSITY OF CALIFORNIA, Oakland, CA (US)

(21) Appl. No.: 16/034,623

(22) Filed: Jul. 13, 2018

Related U.S. Application Data
(63) Continuation of application No. PCT/US2017/013575, filed on Jan. 13, 2017.
(60) Provisional application No. 62/279,653, filed on Jan. 15, 2016.

(51) Int. Cl. A61B 5/00; A61B 5/0402; A61B 5/0476; A61B 8/08; A61B 8/06; A61B 5/03; G06K 9/00; G06K 9/62

(52) U.S. Cl. CPC A61B 5/7267; A61B 5/0402; A61B 5/0476; A61B 5/0075; A61B 8/488; A61B 8/06; A61B 5/7221; A61B 8/5269; G06K 9/00516; G06K 9/6248; G06K 9/6298; G06K 9/6223; A61B 5/031

(57) ABSTRACT

An apparatus and methods for processing monitored biosignals are provided that are particularly suited for reducing noise and artifacts in continuously monitored quasi-periodic biosignals without prior knowledge of the noise distribution. The framework trains a subspace manifold with reference signals. Subsequent signals are successively projected onto the trained manifold and adjusted based on the nearest neighbors of the state of the sample being projected as well as the state of the sample at the previous time point. A denoised or modified output is obtained with inverse mapping. The reference signals may optionally be labeled during manifold training with clinical events/variables or measurable diseases/injuries from a library of relevant labels. During reconstruction, the label of the estimated state in the manifold can be obtained from the label corresponding to the estimated state.

### CROSS-REFERENCE TO RELATED APPLICATIONS

This application is a 35 U.S.C. § 111(a) continuation of PCT international application number PCT/US2017/013575 filed on Jan. 13, 2017, incorporated herein by reference in its entirety, which claims priority to, and the benefit of, U.S. provisional patent application Ser. No. 62/279,653 filed on Jan. 15, 2016, incorporated herein by reference in its entirety. Priority is claimed to each of the foregoing applications.

The above-referenced PCT international application was published as PCT International Publication No. WO 2017/124044 on Jul. 20, 2017, which publication is incorporated herein by reference in its entirety.

### STATEMENT REGARDING FEDERALLY SPONSORED RESEARCH OR DEVELOPMENT

Not Applicable

### INCORPORATION-BY-REFERENCE OF COMPUTER PROGRAM APPENDIX

Not Applicable

### BACKGROUND

### 1. Technical Field

The technology of this disclosure pertains generally to devices and methods for acquiring and processing biosignals, and more particularly to apparatus and methods for reducing noise and artifacts in continuously monitored quasi-periodic biosignals without prior knowledge of the noise distribution. The signal processing framework uses a learned manifold and reconstruction.

### 2. Background Discussion

Biosignal sensing technologies in human physiology are essential in the assessment of the functional state of a patient. Biosignals describe physiological phenomenon that can be continuously measured and monitored by a variety of sensors. All types of physiological mechanisms, systems and biological events generate measurable biosignals that carry information about the mechanisms and events.

Consequently, useful information about the functioning of the body can be extracted from analysis of biosignals. Other than a verbal description of how the patient feels, the only source of information describing the functioning of the human body in healthy and disease conditions may be the acquired biosignals. In addition, information from many different biosignals can be used to understand the status or function of the same organ, specific physiological events or systems.

Sensed biosignals may be static or dynamic, permanent or induced and may have single or multiple parameters. Biosignals can be generated from electrical, mechanical, optical, magnetic, acoustical, thermal and chemical sensors. The ability to obtain accurate and clear information from biosignals depends on the nature and origin of the biosignals and the sensitivity of the measurements. Information is obtained through the process of acquiring measurements to producing signals, signal processing, data analysis, and information interpretation.

However, one significant problem found in many biosignal measurement systems is the presence of noise in the system that can obscure the accurate signal. For example, Intracranial Pressure (ICP) measurements are important in the diagnosis, monitoring and treatment of many vascular brain injuries and diseases. Clinically, ICP is a fundamental physiologic parameter that, if elevated, can lead to a pathological reduction in cerebral blood flow and possible herniation in the brain, resulting in irreversible brain damage or death if left untreated. Currently, the ICP signal is used to diagnose dangerous increases in average pressure.

The standard measurements of the ICP are performed invasively. Although the average ICP is monitored in modern clinical environments, subsequent higher-order analysis on the ICP pulse wave form often requires complex processing, expert annotations, and corrections due to egregious noise introduced during measurements from electronic equipment, electrode transients, displacement of the sensor, or even the patient shifting their posture. Such conditions make it difficult for real-time monitoring software to properly interpret ICP wave form data like that illustrated in FIG. 2.

Noninvasive methods have also been developed to estimate the ICP such as transcranial Doppler (TCD) ultrasound. However, the signals acquired using ultrasound Doppler blood flow instruments are typically contaminated by noise and artifacts due to the nature of the recording system, signal interference, or properties of the blood flow (e.g. turbulence) etc.

The conventional approach to noise reduction is to filter the input signal. When noise is constrained to particular frequencies, such as 60 Hz tonal noise, much of the desired signal can be obtained by applying band stop (notch-type) filters.

In the general case, when a model of the desired signal's spectral content is known, along with an estimate of the noise distribution, adaptive filtering may be used to construct a mean-square optimal filter. However, the problem is more challenging when noise has a spectral density which overlaps significantly with that of the desired signal. Channel estimation is unpredictable in this application, because biosignals are not easily constructed from band-limited primitives. Specifically, it is increasingly difficult to identify a generic noise-floor when the relevant spectral content of the signal is not entirely known.

Another popular approach to the broadband noise problem is to estimate the original signal by assuming some stochastic mixing process. A mixture model uses knowledge of the expected degradation to estimate the most likely values for the originally transmitted signal. When the number of possible source-transmitted symbols is relatively low and discretized, expectation maximization (EM) and maximum a posteriori estimation (MAP) algorithms can be used to estimate the most likely source transmitted symbol, although an initial characterization of both the source and noise distributions is required. In the case of CBFV waveform, the number of possible pulse shapes is not finite and assigning a discrete estimate from a bank of reference signals is not an optimal solution to preserve patient specific features. Moreover, a static characterization of the noise distribution is not always possible in clinical environments, since degradations may vary between sites and may be introduced from a combination of sources (including patient movement, sensor displacement, electronic noise, type of sensor) in varying proportions.

Accordingly, signal filtering approaches in the art have had limited success in producing clear signals. Therefore, there is a need for new signal processing methods for denoising biosignals.

### BRIEF SUMMARY

The present technology provides a signal processing framework that can significantly reduce the amount of noise and artifacts present in a biosignal. The Iterative/Causal Subspace Tracking framework (I/CST) is particularly suited for reducing noise in continuously monitored quasi-periodic biosignals without prior knowledge of the noise distribution. Generally, noise is reduced by reconstructing an estimate of the original signal from a mixture of reference signals. The references are selected by searching the closest neighbors of an input sample in a reduced dimensional space. By tracking the position of consecutive samples in the subspace, causal correction transformations can be iteratively applied to the collected data stream.

The signal processing methods are illustrated in the context of intracranial pressure (ICP) signals to provide a practical demonstration of how it can operate in clinical conditions on routinely acquired biosignals. Nevertheless, the framework is sufficiently generic and provides a platform for applying linear prediction and communication processing of many different biological signals, given the appropriate training parameters.

In this illustration, the ICP signal is tracked using the trajectory of samples on a trained manifold. The procedure is capable of both tracking and actively denoising the ICP signal using a small-signal, differential analysis. Importantly, the framework uses shape, time, and elevation constraints when constructing the manifold. These constraints enable a strong localization of projections in a hyper-volume for ICP pulses localized in time, elevation, and morphology.

Then, even naive vector quantization algorithms can provide a significant improvement in the signal to noise ratio. In addition, tracking the trajectory of samples in the subspace can enable more advanced linear and non-linear prediction algorithms that may be capable of predicting the ICP elevation minutes prior to its onset. Considering the critical state of most patients being monitored, the ability to identify elevations prior to their onset is particularly useful.

Generally, the signal processing has a three part framework: 1) acquiring the input/reference biosignals; 2) preparing and training the subspace manifold which represents the data in a modified space; and 3) the output of the manifold.

In this illustration, the input at part 1) can be a biosignal such as a

CBFV waveform acquired using transcranial doppler (TCD) or other waveform such as an ICP waveform or an ICP elevation (or any surrogate measure known to correlate with CBF). The output of iteratively projecting noisy signals on the trained manifold in part 3) would be the denoised CBFV waveform, denoised ICP waveform, denoised ICP elevation (and any other metric that has been associated during the training of the manifold).

Although the processing methods are described in the context of ICP patients, it will be understood that the framework can be trained and used on different populations such as TBI patients as well. Since the framework involves the sequential estimation of a state in the manifold that is linked to the properties of the original reference (e.g. CBFV) waveform, the state is therefore related to any conditions that are known to be associated to a change in the reference biosignal state (CBFV).

For example in this illustration, the manifold could also be trained to output any of the following conditions/applications: CBFV waveform/level denoising; ICP waveform/level denoising; CBFV waveform/level forecast; ICP waveform/level forecast; Estimation of ICP level from CBFV waveform; CBFV/ICP signal assessment (current TCD methods are user dependent so there is a need to assess the quality of the data); Assessment of collateral circulation and reperfusion from CBFV; Estimation of infarct volume in acute stroke; Detection of stenosis; Presence of CBF regulation dysfunction due to TBI; Detection of repercussion injury; Evaluation of CVR test; Evaluation of intravascular treatment, and the detection of vasospasms.

In one embodiment, the framework is formalized as a dynamic Markov model that uses manifold learning to represent the temporal joint distribution between the following components:

(A) CBFV waveform (at the beat level);

(B) ICP waveform (at the beat level); and/or

(C) Elevation of ICP (or any surrogate continuous measure correlated with ICP or CBFV waveform).

In another embodiment, the system is trained from previously labeled data samples to learn the complex relationship between time-aligned CBFV beats, ICP beats, and ICP elevation using a machine learning method based on nonlinear subspace analysis. At each time t, the model represents 2 state variables that are observed (they correspond to the noisy observations of A and B) and 2 counterparts that are hidden (they correspond to the true, unobserved values of A and B). The hidden and observable states are linked through a Gaussian likelihood function. The model learns the relationship between A, B, and C using manifold learning which is then represented nonparametrically using a kernel density estimation. Denoising is posed as inference in the nonparametric Bayesian model.

Current methods of CBFV and ICP signal denoising have not used the temporal relationship between successive beats and the intrinsic relationship that exists between the ICP level and the waveforms. Beneficially, the technology represents the complex temporal relationship between CBFV waveforms, conditions/applications such as ICP waveforms, and ICP elevation as well as others mentioned above and uses this representation to constrain the denoising problem. Advantages include (a) significant decreases of noise and artifacts in the signal without losing pertinent morphological features; and (b) once trained, the system can be used on individual inputs only (or on any combination of inputs) e.g. CBFV, ICP waveforms, ICP level and/or other physiologic biosignals such as CVR regulation.

In another embodiment, the technology may be implemented into a TCD acquisition device or a bedside monitor. In another embodiment, the technology may be integrated to a TCD recording device for real-time denoising of CBFV waveforms. In some embodiments, the device or monitor measures a biosignal which is then evaluated using the trained manifold.

According to one aspect of the technology is to provide an apparatus and system that builds a manifold based on prior measurements and then apply it to obtain outputs from new, successive measurements. The new successive measurements may be made in real time from a device or monitor.

Another aspect of the technology is to provide a processing framework that trains a subspace manifold based on reference signals and labels of measurable condition variables and then iteratively projecting new signals on the trained manifold graph that are then reconstructed.

Another aspect of the technology is to provide a system and method for significantly reducing the amount of noise and artifacts present in one or more biosignals and allowing the estimation of physiological conditions.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a functional flow diagram of the methods for denoising biosignals according to one embodiment of the technology.

FIG. 2 is a plot of a typical intracranial pressure signal (ICP) recorded in clinical conditions. ICP typically exhibits significant noise on its envelope that challenges its morphological analysis.

FIG. 3A is a plot of the average and standard deviation of pulsatile ICP clusters at each time point relative to hypertension onset.

FIG. 3B is a plot showing the morphology of time-localized ICP clusters illustrated with three groups for the baseline, transition, and hypertensive regions.

FIG. 4 is a projection of a set of ICP beats onto the first two dimensions (ω₁ and ω₂) of the manifold. Each dot represents a 3-sec cluster extracted from the training set. The transition from normal (left side) to elevated (right side) ICP is depicted that represents the temporal-index relative to the elevation plateau.

FIG. 5A is a projection of the current sample onto a subspace defined by a training constellation.

FIG. 5B is a graph showing constraints of the projection to a mixture of the k-nearest neighbors of the previous sample.

FIG. 5C is a plot showing the final estimate is a smooth signal which demonstrates significant improvements over generic filters shown in FIG. 5D in terms of smoothness, mean-square error, and distinguishable morphology such as the number and location of local maxima and minima.

FIG. 5D is a plot of a waveform using a generic filter for comparison.

FIG. 6A is a graph showing signal to noise ratio for varying levels of additive white gaussian noise (AWGN).

FIG. 6B is a graph showing signal to noise ratio for varying levels of FIG. 6B varying levels of Poisson noise magnitudes.

FIG. 7A is a graph depicting denoising of an idealized ICP stream corrupted by AWGN noise of variance σ=4% at time +3 min.

FIG. 7B is a graph depicting denoising of an idealized ICP stream corrupted by AWGN noise of variance σ=4% at time −5 min.

FIG. 7C is a graph depicting denoising of an idealized ICP stream corrupted by AWGN noise of variance σ=4% at time −19 min.

FIG. 7D to FIG. 7F are graphs at the same time points shown in FIG. 7A. Signals were projected to the subspace in FIG. 7A, and denoised using the RS01 algorithm using geodesic constraints. FIG. 7D at t=−19 min. FIG. 7E at t=−5 min, and FIG. 5F at t=+3 min.

FIG. 7G is a graph of corresponding estimates compared to various generic filters at t=−19 min.

FIG. 7H is a graph of corresponding estimates compared to various generic filters at t=−5 min.

FIG. 7I is a graph of corresponding estimates compared to various generic filters at t=+3 min.

### DETAILED DESCRIPTION

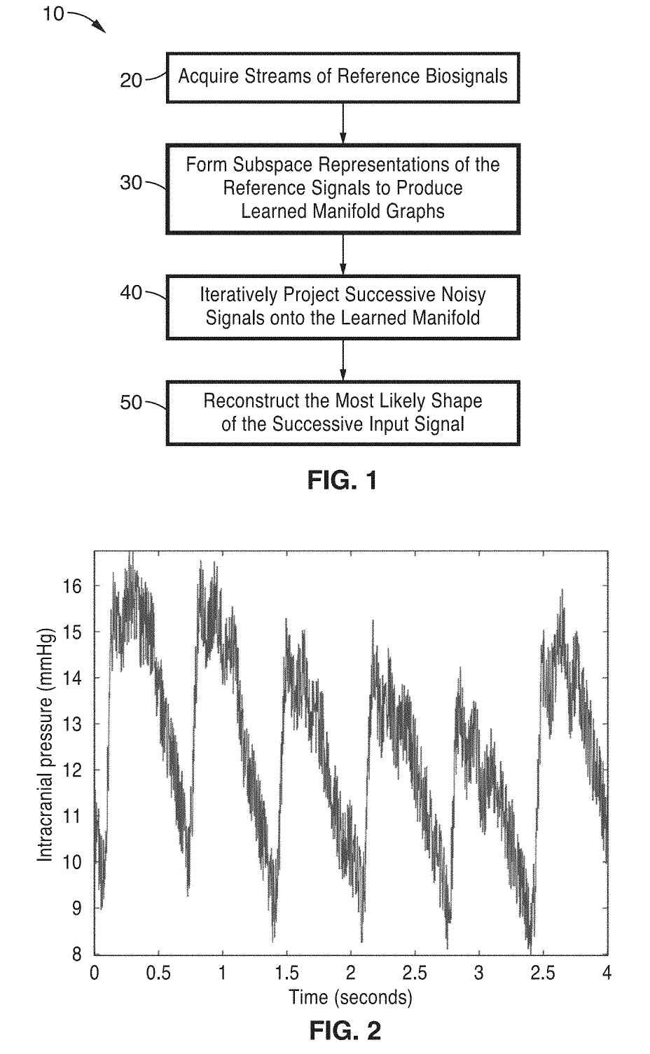

Referring more specifically to the drawings, for illustrative purposes, embodiments of systems and methods for biosignal processing are generally shown. Several embodiments of the technology are described generally in FIG. 1 through FIG. 7I to illustrate the signal processing system and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

Turning now to FIG. 1, a flow diagram of one embodiment of an Iterative Causal Subspace Tracking (I/CST) method 10 for performing biosignal processing illustrated within the context of denoising ICP related biosignals is generally shown. The processing framework is used to process a quasi-periodic input signal into a set of successive components (e.g. related to the heartbeat in the case of ICP and CBFV) that are successively projected onto a manifold representation (a manifold is a previously learned space that hold data samples in a structured, spatially smooth way). The projection onto the manifold is adjusted based on the nearest neighbors of the state of the sample being projected as well as the state of the sample at the previous time point. Once the sample is adjusted to its new position, an inverse mapping process can be applied to the original input space to obtain a denoised/modified output. The inverse mapping can also be applied to obtain the labeling data (if any) provided during the training of the manifold. The framework can significantly reduce the amount of noise and artifacts present in the signal.

At block 20 of FIG. 1, one or more biosignals are selected and streams of reference signals are acquired with conventional sensing devices. Examples include electrocardiogram (ECG), transcranial Doppler (TCD), electroencephalogram (EEG), near infrared spectroscopy (NIRS) as well as generic pulsatile signals such hormone or protein sensors.

A learned/trained subspace manifold is then produced at block 30 of FIG. 1. The primary objective of the framework at block 30 is to learn a manifold which is a subspace representation of the reference data that is represented as a graph and on which consecutive noisy pulses can be projected and denoised continuously at block 40 and block 50. A wide variety of manifold learning techniques exist, such as ISOMAP, Laplacian eigenmap, locally linear embedding, that could be suitable for use with pulsatile data such as ICP. In some embodiments, consecutive pulses are recorded and analyzed in real time or during the course of the measurement of biosignals by a device or monitor such as a TCD system.

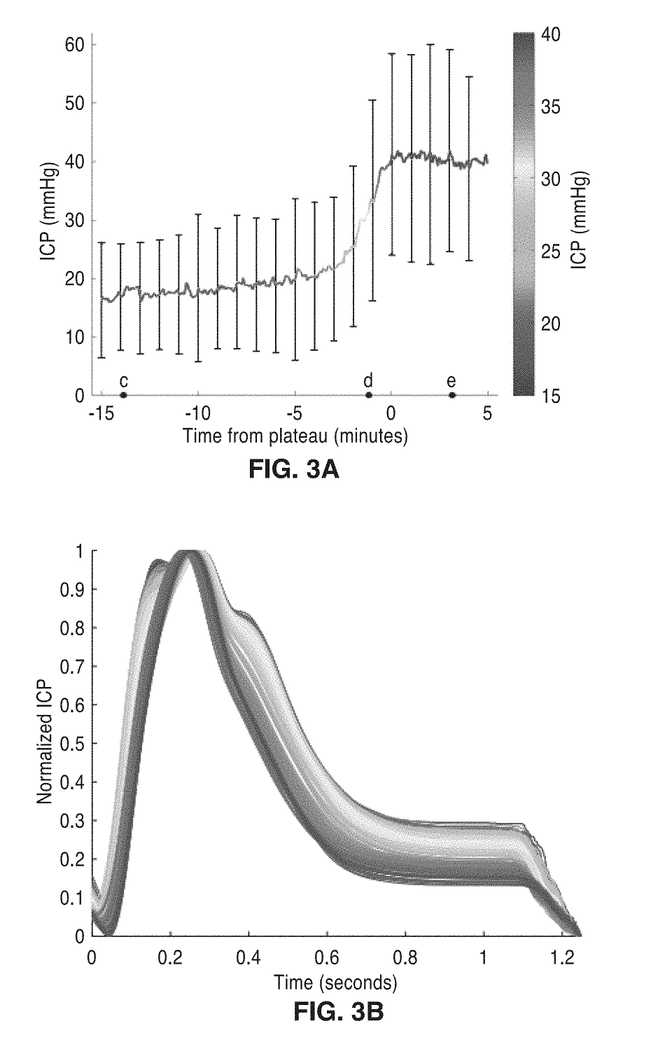

However, one important element of the processing framework is to constrain the learning of the manifold to take into account the average ICP of the pulses and their relative position in time, which are related to the overall shape of the pulse as can be seen in FIG. 3B, for example. By doing so, pulses with similar shapes and ICP, and close in time will also be close to each other in the manifold representation. Because the manifold will be used to track pulses over time, it is important that similar shapes of pulses remain close to each other for optimal reconstruction. The use of a graph representation allows the geodesic distance to be computed (i.e. distance in the graph, as opposed to Euclidean distance), from which variations between signals can be quantified more easily.

During training, the (I/CST) process utilizes a subspace learning procedure followed by the construction of a graph defined on that space. Once learned, the graph manifold can then be used by the tracking procedure at block 40 to iteratively project successive noisy pulses on the graph, in order to reconstruct their most likely shape in the original input space.

One preferred subspace learning procedure at block 30 obtains the subspace manifold using a kernel discriminant analysis (KDA) of reference pulses, which is solved using a spectral regression (SR) framework. The goal of SR-KDA in this case is to find a regression model which leads to similar subspace projections yᵢ∈Y for input data samples (i.e. pulses) xᵢ∈X that are morphologically similar.

SR-KDA utilizes a graph representation of the data where each vertex represents a data point. An affinity matrix W∈R^{m×m} is preferably used to represent the graph and associates a similarity weight Wᵢⱼ to each edge {i, j}; given a set of m samples x_{i=1, . . . , m}. A graph embedding technique is used to represent each vertex of the graph as a vector sᵢ∈S that preserves similarities between the vertex pairs, where similarity is measured by the edge weight. To obtain an optimal graph embedding, the objective is to ensure that samples that are close to each other in the graph are also close in the subspace representation. This can be achieved by minimizing the following measure ∈:

$$\begin{matrix}∈ = {\sum }_{i , j = 1}^{m} {( {s}_{i} - {s}_{j} )}^{2} \cdot {W}_{i , j} \\ = 2 {S}^{T} LS\end{matrix}$$

where L=DW is the graph Laplacian and D is a diagonal matrix whose entries are column sums of W. The optimal S can be obtained by finding the largest k generalized eigenvectors λ of the eigen problem:

WS=λDS

Once the eigen eigenvectors λ are computed, the embedding S of the data can be used as labels, and the regression problem solved as a standard ridge regression:

$${arg}_{a} min {\sum }_{i = 1}^{m} {( {a}^{T} {x}_{i} - {s}_{i} )}^{2} + α {\sum }_{i = 1}^{m} {a}_{j}^{2}$$

As mentioned earlier, the preferred framework incorporates constraints on the learning of the subspace to take into account the average ICP of the pulses and their relative-time position. This may be done via the construction the W matrix so that the weights reflect a weighted distance between the respective pulse waveform p, ICP c, and time index t. Specifically:

$${G}_{1} ( i , j ) = {w}_{p} p + {w}_{c} c + {w}_{t} t$$

$${G}_{2} ( i , j ) = \{ \begin{matrix}1 & if {G}_{1} ( i , j ) ∈ knn ( {G}_{1} ( i , : ) , k ) \\ 0 & otherwise\end{matrix} \\ W = {G}_{1} ⊙ {G}_{2}$$

where wₚ, w_{c}, and wₜ represent the weigh associated with the different input modalities respectively, represents the element-wise multiplication of matrices, and the value k=5 was chosen empirically. Multiplication by the mask G2 constrains the association of a pulse on the manifold to its k-nearest neighbors in the mixed-modality input space.

The tracking on the manifold process at block 40 of FIG. 1 should be able to handle various levels of noise. When the noise envelope is small, the locality-preserving properties of the SR-KDA embedding ensure a projection to areas on ably small hyper-volume around the expected point. Conversely, when the noise envelope is large there is no guarantee of locality in the graph. Since the framework should be independent of the noise distribution (for applicability in clinical scenarios), a method of constraining the projection of consecutive pulses to nearby locations is preferred.

Since consecutive ICP pulses are likely to exhibit similar shapes, the trajectory of consecutive samples on the manifold is used as a general constraint for the denoising process. The trajectory may be obtained by projecting consecutive samples into the learned subspace. Sequential tracking is then applied to estimate the most likely coordinates of the successive samples in the subspace. The coordinates are then reconstructed back to the input space using an inverse mapping to produce the denoised waveform at block 50. In essence, the procedure achieves complex non-linear predictions by employing simple prediction algorithms in the reproducing kernel Hilbert space.

Of all possible prediction algorithms, perhaps the most simple and understandable is the k-nearest neighbor regression. In this algorithm, a value is constrained to the average of the values of its k-nearest neighbors. In the case of ICP signals, this translates to the average of similar waveforms, where the notion of similarity is measured using distance in the computed graph. This procedure was formalized and adapted to the I/CST framework as (RS01) denoising algorithm.

Specifically, RS01 uses an inversely weighted reconstruction at block 50 to estimate the expected signal waveform. RS01 is designed to improve the signal quality of continuous pulsatile signals (such as ICP) existing in R^{N} by exploiting the locality-preserving properties of the SR-KDA embedding in the subspace Y3.

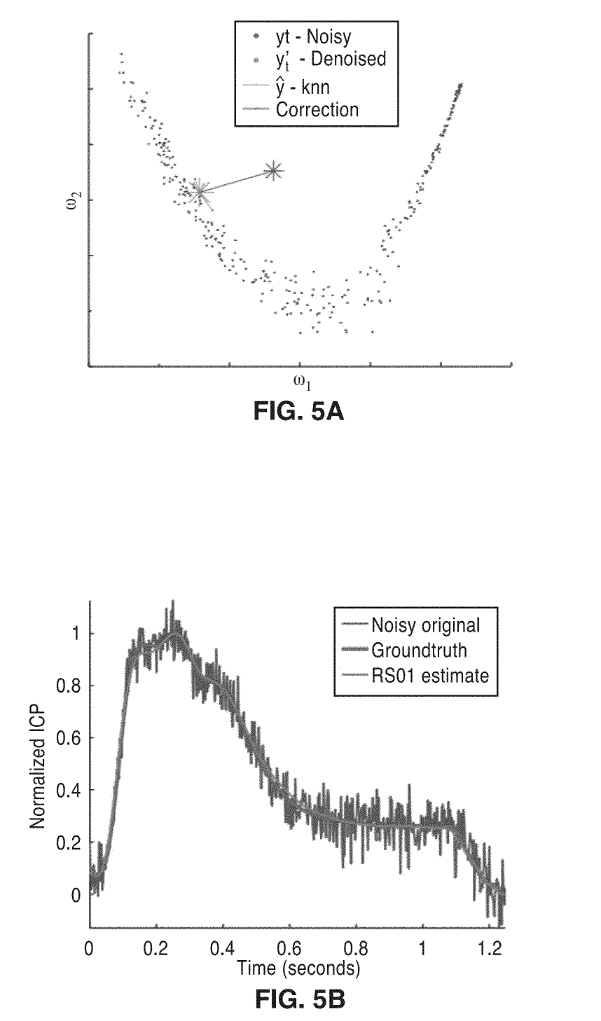

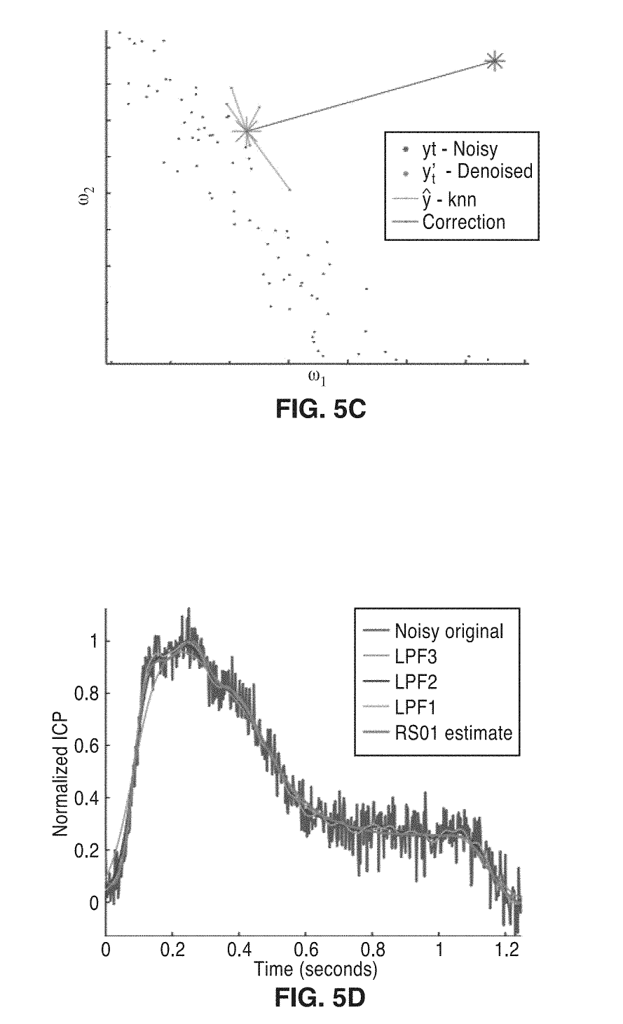

The algorithm uses graph searching to find the k-nearest neighbors of noisy samples in a provided subspace and computes an estimate of the expected signal using a mixture of waveforms. In order to enforce locality, the k-nearest neighbors of the previous time-sample are used rather than those corresponding to the current time-sample, thereby constraining the projection to nearby locations (See e.g. FIG. 5A to FIG. 5D). RS01 also computes an error signal indicating the level of confidence in the real-time sample based on past input. The error signal is useful for the identification of large fluctuations in noise, as well as sudden deviations in signal properties (e.g. morphology).

One of the main benefits in this design is that the trajectory (i.e. a directed path on a graph) of consecutive samples can be used to augment tracking and analysis algorithms. Furthermore, I/CST's ability to operate hierarchically on N-dimensional observations in real-time, as opposed to retrospectively, provides a robust platform for developing automated adaptive software systems, including control and learning systems which rely on programmed routines but require live observation-based triggers.

Accordingly, signals, such as ICP, can be cast into an arbitrary discriminative domain where previously developed elementary algorithms are still effective. In particular, by reducing the dimensionality of the subspace where signals are projected, the operation of such algorithms is considerably improved (both in complexity and accuracy) due to the low dimensionality of the search hyper-volume. This result can be exploited by medical and biological analysis software for the purpose of state-prediction and active denoising.

It can be seen that the I/CST methods can be adapted to denoise generic signals (i.e. processing without domain-specific knowledge) by first employing morphological clustering and subsequently learning the subspace with continuous annotations. The morphological clustering can be domain-specific, but it is not necessary. A method of N-dimensional k-means clustering and regression could be applied to identify several possible distinct symbols which vary proportionally with some statistic (mean, variance, kurtosis, etc), although an appropriate clustering procedure is non-trivial. Obviously, the statistics used should be relevant to the waveforms being compared for optimal performance, but this criteria is not necessary for the basic operation of the process. In this sense, the I/CST framework is sufficiently general and by itself does not require any domain-specific knowledge. As such, I/CST can be easily extended to other quasi-periodic biosignals given an appropriate context specific clustering and comparison strategy. Therefore, the data may originate from different subject populations.

The reference signals may optionally be labeled during manifold training with clinical events/variables or measurable diseases/injuries from a library of relevant labels. During reconstruction, the label of the estimated state in the manifold can be obtained by just looking up the label corresponding to the estimated state. For example, the list of labels that can be associated with CBFV includes: degree of collateral blood flow circulation to the brain, quality of reperfusion after reperfusion therapy, lesion volume in acute stroke and TBI, degree/presence of stenosis, presence of CBF regulation dysfunction due to TBI, presence of reperfusion injury, result of cerebral vascular reactivity (CVR) test, degree of success of intravascular treatment, severity of vasospasms.

As an illustration, the input reference signals could be the CBFV waveform. The labels applied during training could be ICP level, a numerical clinical variable or categorical clinical variable and the modified outputs may range from denoised CBFV waveforms, ICP levels and waveforms and numerical or clinical variables.

Finally, because the temporal information is modeled in the framework, it can be used in three modes: smoothing, filtering, and prediction; as they respectively correspond to the modified output in the past, present, and future.

The technology described herein may be better understood with reference to the accompanying examples, which are intended for purposes of illustration only and should not be construed as in any sense limiting the scope of the technology described herein as defined in the claims appended hereto.

### Example 1

In order to demonstrate the operational principles of the apparatus and signal processing methods, a dataset of ICP and electrocardiogram (ECG) signals were recorded for a total of 70 patients who were being treated for various intracranial pressure related conditions including idiopathic intracranial hypertension, Chiari syndrome, and slit ventricle patients with clamped shunts was acquired and processed using the processing steps shown generally in FIG. 1.

The ICP of each patient was sampled continuously at 400 Hz using an intraparenchymal microsensor placed in the right frontal lobe. Intracranial hypertension (IH) episodes were identifies and the time of the elevation onset, elevation plateau, and invasive cerebrospinal fluid drainage were annotated. Using these annotations, 20-minute segments, capturing the transition from a state of normal (0 to 20 mmHg) to elevated ICP (>20 mmHg), were extracted as reference data. The segments were time-aligned such that they contained 15 min of data before the plateau and 5 min after.

Individual ICP pulses were extracted from the recorded segments using a correlation of ICP with R-wave peaks in the ECG signal. Because this method was dependent only locally on the R-wave peaks, the segmentation was sufficiently accurate and largely invariant to heart-rate variability. The extracted pulses were distilled into 3 variables: (1) amplitude and length normalized vectors containing pulsatile information, (2) mean value of the original pulse, and (3) starting time-index of the pulse relative to the elevation plateau.

An idealized ICP signal was then generated by accumulating the beats that occurred at similar relative time intervals (i.e. by binning the pulses falling within every 3 sec interval) and computing their average. The resulting average signal which still holds pulsatile information is shown in FIG. 3A and was designated the ground truth. FIG. 3A is a plot of the average and standard deviation of pulsatile ICP clusters at each time point relative to hypertension onset. FIG. 3B is a plot showing the morphology of time-localized ICP clusters illustrated with three groups for the baseline, transition, and hypertensive regions. The average shape of the ICP waveform is related to the ICP elevation as illustrated in FIG. 3B. Pulses corresponding to normal ICP tend to exhibit three peaks (left side), while higher ICP ones generally tend to become unimodal (right side).

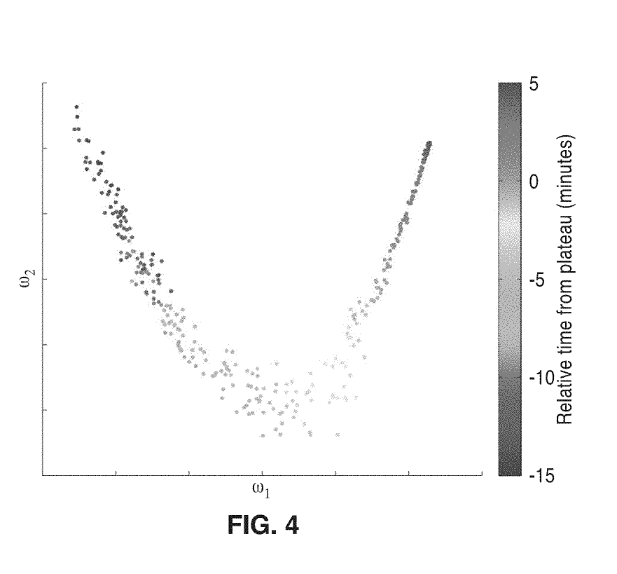

The subspace learning algorithm was then applied to construct and train a suitable graph manifold that was used thereafter by the tracking algorithm to iteratively project successive noisy pulses onto the graph and refine their position in the learned manifold. As shown in FIG. 4, a set of ICP beats were projected on to the first two dimensions (ω₁ and ω₂) of the manifold. Each dot represents a 3 second cluster extracted from the training set.

Projection of consecutive samples into the learned subspace allowed the estimation of coordinates of successive samples on the manifold. The coordinates are then reconstructed back to the input space using an inverse mapping to produce the denoised waveform. As shown in FIG. 5A, the current samples were projected onto the subspace defined by a training constellation. The projections were constrained to a mixture of the k-nearest neighbors of the previous sample as seen in the graph of FIG. 5B. The final estimate seen in FIG. 5C is a smooth signal. As illustrated in FIG. 5D, this signal is a significant improvement over generic filters in terms of smoothness, mean-square error, and distinguishable morphology such as the number and location of local maxima and minima.

The methodology that reconstructs a likely estimate of the original signal in noisy scenarios using a mixture of waveforms was confirmed. By searching the k-nearest neighbors of a sample in the learned subspace, the process effectively constrained and denoised the analog estimate. The benefit of such a scheme was demonstrated by significant increases in the average and peak SNR of a 20-min ICP recording.

Organic signals, such as ICP, tend to exhibit a range of features that are locally temporally correlated, but vary continuously with the waveform. In this light, the goal was to develop a robust information-processing system for analog biosignals. The signal processing methods capture the continuously varying characteristics of ICP waveforms. This was accomplished by characterizing the morphology of ICP waveforms via clustering, warping the subspace using continuous valued statistics (DC value and expert annotations), and tracking the progression of waveforms within the proposed I/CST framework.

### Example 2

To further demonstrate the effectiveness of the I/CST framework, the ability to effectively track input waveforms that have been degraded by various levels of noise was tested. In particular, the framework was tested with additive white-Gaussian noise and Poisson noise, although the method is equally valid for artifact detection via the error signal. The noise testing procedure was comprised of four steps: (1) degrade the original ground truth signal with the selected noise profile, (2) apply the selected denoising kernel, (3) measure the SNR of each beat, and (4) average the SNR over the entire ICP signal.

The evaluation strategy compared the signal-to-noise ratio (SNR) of input waveforms (baseline) to those produced by various denoising kernels. The goal of these kernels was to remove the noise envelope from the degraded signal and to return the original pressure signal. In this context, noise was defined as any deviation from the true waveform, and was typically reported by magnitude (e.g. 2-norm). The SNR was calculated on a beat-by-beat basis.

In the evaluation, the ground truth data generated from the 70 IH episode dataset of Example 1 was used to synthesize noisy signal streams representing various levels of degradation. AWGN was typically parametrized by the parameters μ=0 and σ, which represent the mean and standard deviation of the distribution. As such, a zero-mean Gaussian distribution was sampled and its variance scaled relative to the normalized ICP pulse amplitude, max x_{n×1}. This noise profile was applied additively to the full length of the ICP signal to generate noisy testing data. Similarly, Poisson noise is typically parametrized by the parameter λ which represents both the mean and variance. To generate different levels of Poisson-noise, the original ICP stream was used as the mean, while the variance was similarly scaled relative to the normalized ICP pulse amplitude.

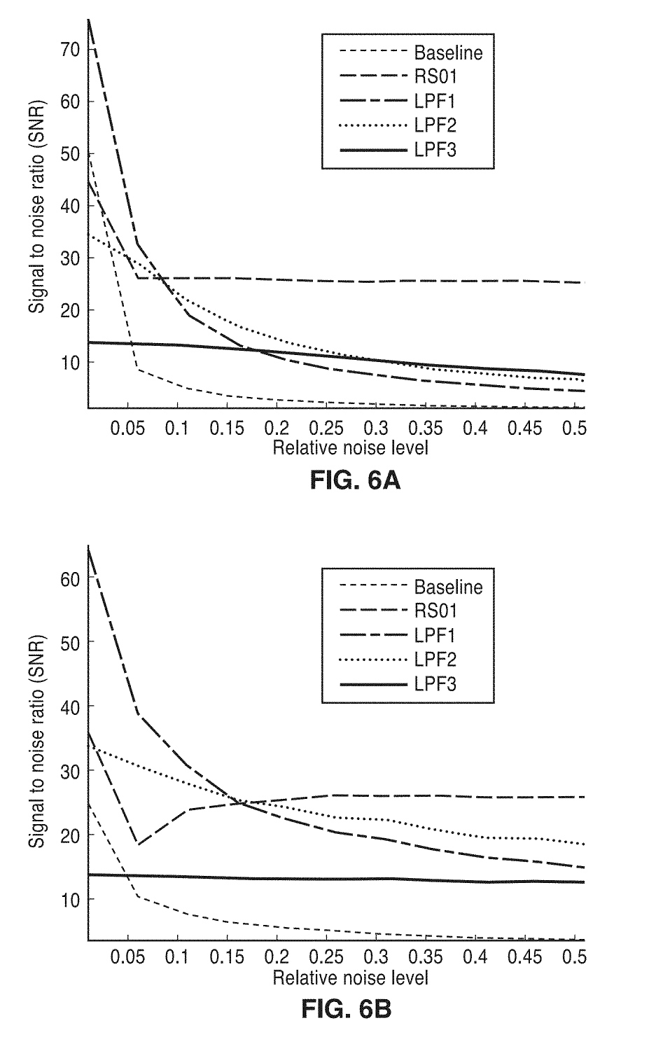

In the tests, the results of the denoising procedure (RS01) was compared with the results of various Gaussian low-pass filters (LPF) as seen in FIG. 6A and FIG. 6B. These generic convolutional filters (kernels) were constructed by generating several ideal zero-mean Gaussian distributions with variances corresponding to different levels of noise. The value of variance was scaled relative to the normalized ICP amplitude, and the length of the kernel was chosen to be sufficiently large to avoid edge effects during convolution. In the tests, the methods were compared against three such generic kernels, termed LPF1, LPF2, LPF3, corresponding to variances of 5%, 10%, and 20% of the normalized ICP amplitude.

Although the generic filters do provide some smoothing, it was shown that even the simplest I/CST implementation provides a significantly more desirable result over filtering. Over all the tests, the tested processing methods performed consistently well with an average SNR improvement of 758% and 396% over AWGN and Poisson noise respectively. The average SNR was computed from experiments with noise variances uniformly distributed on 5% to 35% of the normalized beat amplitude.

One important feature of the tested RS01 procedure is that it does not require knowledge of the noise profile to be effective. Although such information can be useful to effectively clean up the signal, the typical convolutional approach is limited for two reasons: (1) the size of the averaging kernel is not easily determined directly from the input data, making it impractical without proper calibration or channel-estimation protocols, and (2) convolution in the time domain corresponds to a multiplication in the frequency domain, so typical Gaussian filters will effectively mask potentially-useful high-frequency information.

The results shown in FIG. 6A and FIG. 6B demonstrate this via a dramatic increase in the SNR, particularly at higher noise powers. RS01 significantly outperformed typical low-pass filters when evaluated across various noise magnitudes. This result was elucidated by the variable SNR performance of any-single LP filter. From these tests, it was evident that no single generic filter could be unanimously selected for denoising since its performance depends on the level of degradation, which is essentially unpredictable in clinical scenarios. In contrast, RS01 pays a small penalty in initially but performed consistently well across several noise levels, making it a practical tool for clinical data collection and analysis.

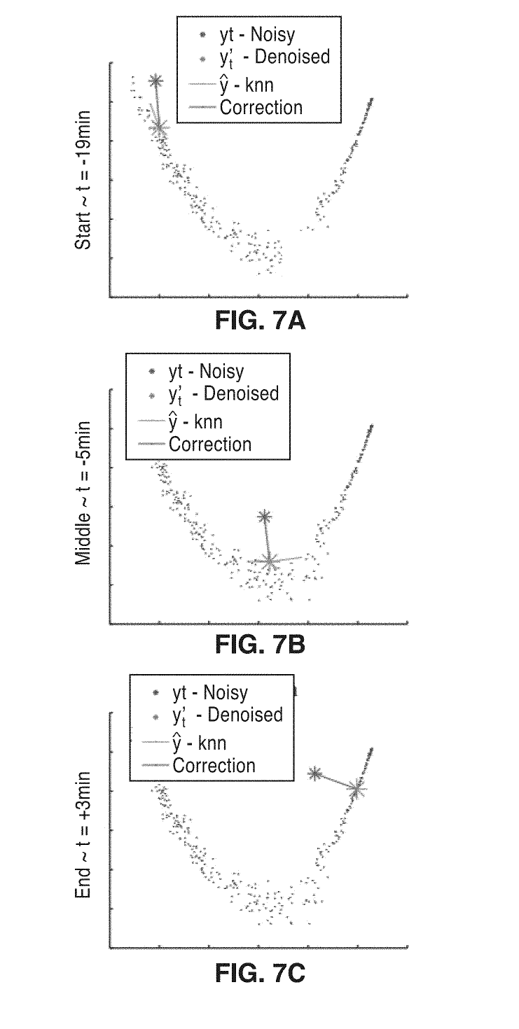

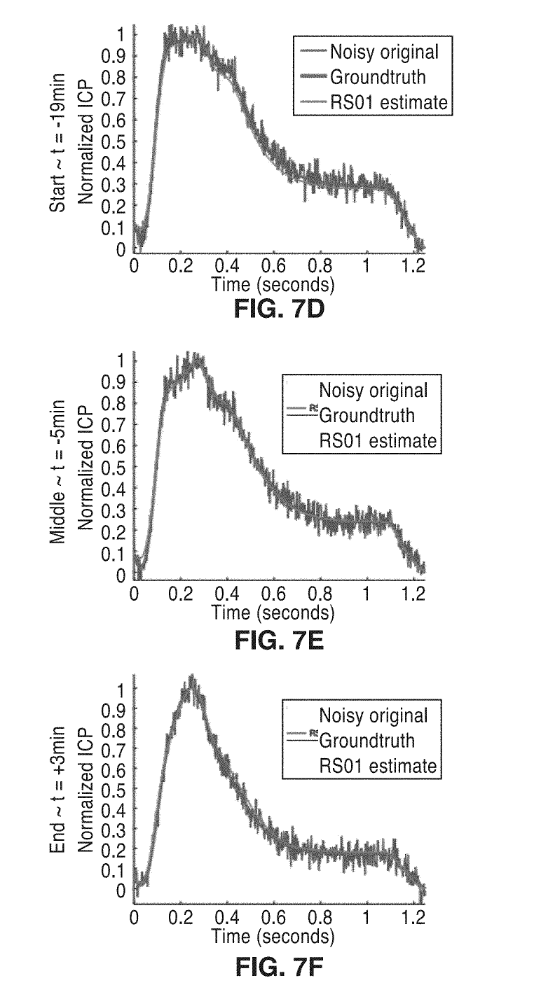

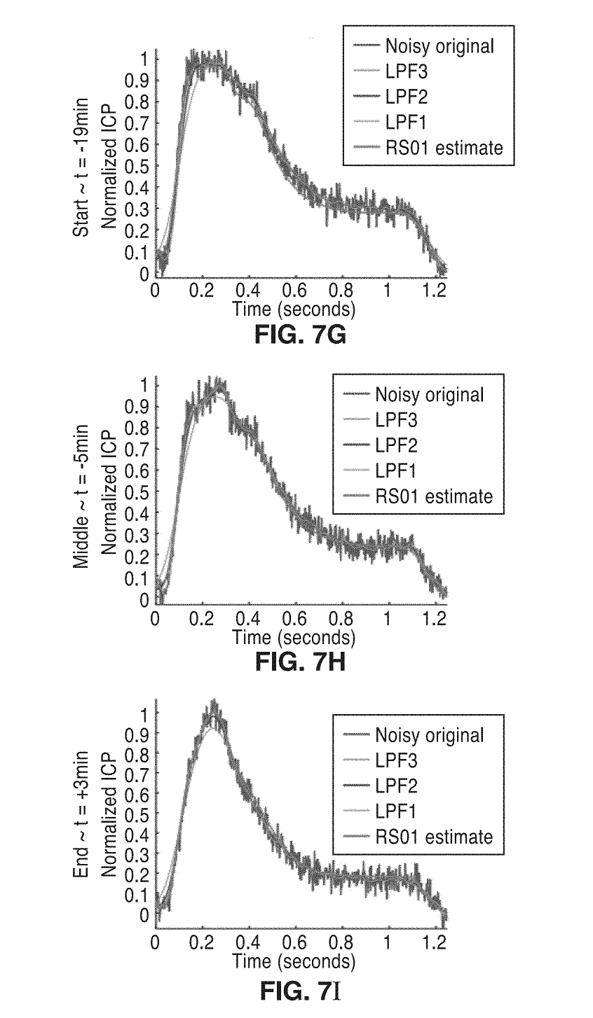

The power of the tested procedure RS01 is evident both quantitatively from the SNR computation, and qualitatively by visualizing the denoised waveform is also shown in FIG. 7A through FIG. 7I. FIG. 7A through FIG. 7C show the denoising of an idealized ICP stream corrupted by AWGN noise of variance σ=4% with the signals projected to the subspace at different time points. FIG. 7A is a graph at t=−19 min, FIG. 7B is a graph at t=−5 min and FIG. 7C is a graph at t=+3 min. Corresponding waveforms denoised using the RS01 procedure with geodesic constraints at the t=−19 min time points is shown in FIG. 7D, at the t=−5 min time point in FIG. 7E and at the t=+3 min time point in FIG. 7F. Finally, the computed estimate at the corresponding time points is compared to various generic filters in FIG. 7G through FIG. 7I.

The tested procedure (RS01) consistently provided a smooth signal, while various time-domain filters do not guarantee such a criteria particularly at the boundaries of each pulse-beat. Furthermore, generic filters expose several local minima and maxima, which reduces the accuracy of subsequent higher-order processing algorithms, such as MOCAIP, while the procedure seems to match both the number, location, and amplitude of salient waveform peaks. In this respect, the tested process kernel provided a result that was even better than that suggested by the 2-norm errors.

The observed resilience of RS01 process to high-levels of noise (particularly AWGN) can perhaps be attributed to the averaging characteristic of the chosen mapping metric (Euclidean distance). Because deviations at any position in the signal contribute to the error during morphological comparisons, the error can be minimized by choosing waveforms that match the natural oscillations present in the noisy signal.

It can be seen that the I/CST procedure provides a convenient framework to improve the quality and analysis of measured biosignals by providing a mechanism for triggered analysis and qualification of noisy samples. The benefit of this procedure is that new samples can be projected in real-time, and subsequent analysis can be performed in parallel to qualify the trajectory and properties of projected nodes.

From the description herein, it will be appreciated that that the present disclosure encompasses multiple embodiments which include, but are not limited to, the following:

1. An apparatus for reducing noise in continuously monitored quasi-periodic biosignals without prior knowledge of the noise distribution, comprising: (a) a computer processor; and (b) a non-transitory computer-readable memory storing instructions executable by the computer processor; (c) wherein the instructions, when executed by the computer processor, perform steps comprising: (i) providing a one or more reference signals; (ii) forming a subspace representation of the reference signals to produce a learned manifold graph; (iii) iteratively projecting successive signals on the learned manifold graph; and (iv) reconstructing the most likely shape of the successive signal.

2. The apparatus of any preceding embodiment, wherein the instructions when executed by the computer processor further perform steps comprising: extracting individual pulses from the plurality of reference signals; distilling at least one variable from the extracted pulses; normalizing the extracted pulses; and clustering similar normalized pulses to produce an idealized reference signal.

3. The apparatus of any preceding embodiment, wherein the reference and successive signals are signals selected from the group consisting of electrocardiogram (ECG) signals, transcranial Doppler (TCD) signals, electroencephalogram (EEG) signals, and near infrared spectroscopy (NIRS) signals.

4. The apparatus of any preceding embodiment, wherein the instructions when executed by the computer processor further perform steps comprising: acquiring a cerebral blood flow velocity (CBFV) waveform as a reference signal from a transcranial doppler (TCD) waveform, an ICP waveform, and an ICP elevation.

5. The apparatus of any preceding embodiment, wherein the subspace is obtained by a kernel discriminant analysis (KDA) of the reference signals solved using a spectral regression (SR) framework.

6. The apparatus of any preceding embodiment, wherein the reconstructing of the successive signal comprises: estimating likely coordinates of successive samples in subspace with sequential tracking; and reconstructing the estimated coordinates back into input space using inverse mapping to produce a denoised waveform.

7. The apparatus of any preceding embodiment, wherein the inverse mapping comprises: searching the k-nearest neighbors of a sample in the learned subspace, wherein the waveform estimate is effectively constrained and denoised.

8. The apparatus of any preceding embodiment, wherein the instructions when executed by the computer processor further perform steps comprising: associating a label with measurable physiological conditions correlated with states of the reference biosignals; and labeling reference signal states with at least one label from a library of labels.

9. The apparatus of any preceding embodiment, wherein the library of labels comprises labels associated with cerebral blood flow velocity (CBFV) selected from the group consisting of degree of collateral blood flow circulation to the brain, quality of reperfusion after reperfusion therapy, lesion volume in acute stroke and traumatic brain injury, degree/presence of stenosis, presence of cerebral blood flow regulation dysfunction due to traumatic brain injury, presence of reperfusion injury, result of cerebral vascular reactivity (CVR) test, degree of success of intravascular treatment, and severity of vasospasms.

10. The apparatus of any preceding embodiment, wherein the instructions when executed by the computer processor further perform steps comprising: assessing the quality of a signal by computing a difference between the denoised waveform and the original reference waveform; wherein the larger the difference between signals, the lower the quality of the original signal.

11. A computer implemented method for reducing noise in continuously monitored quasi-periodic biosignals without prior knowledge of the noise distribution, the method comprising:(a) acquiring a plurality of reference signals; (b) forming a subspace representation of the reference signals to produce a learned manifold graph; (c) iteratively projecting successive signals on the learned manifold graph; and (d) reconstructing the most likely shape of the successive signal; (e) wherein the method is performed by a computer processor executing instructions stored on a non-transitory computer-readable medium.

12. The method of any preceding embodiment, wherein the reference and successive signals are signals selected from the group consisting of electrocardiogram (ECG) signals, transcranial Doppler (TCD) signals, electroencephalogram (EEG) signals, and near infrared spectroscopy (NIRS) signals.

13. The method of any preceding embodiment, further comprising: extracting individual pulses from the one or more reference signals; distilling at least one variable from the extracted pulses; normalizing the extracted pulses; and clustering similar normalized pulses to produce an idealized reference signal.

14. The method of any preceding embodiment, wherein the subspace is obtained by a kernel discriminant analysis (KDA) of the reference signals solved using a spectral regression (SR) framework.

15. The method of any preceding embodiment, wherein the reconstructing of the successive signal comprises: estimating likely coordinates of successive samples in subspace with sequential tracking; and reconstructing the estimated coordinates back into input space using inverse mapping to produce a denoised waveform.

16. The method of any preceding embodiment, wherein the inverse mapping comprises: searching the k-nearest neighbors of a sample in the learned subspace; wherein the waveform estimate is effectively constrained and denoised.

17. The method of any preceding embodiment, further comprising: associating a label with measurable physiological conditions correlated with states of the reference biosignals; labeling reference signal states with at least one label from a library of labels; and estimating signal state in the manifold from the label during reconstruction.

18. The method of any preceding embodiment, further comprising: assessing the quality of a signal by computing a difference between the denoised waveform and the original waveform; wherein the larger the difference between signals, the lower the quality of the original signal.

19. A computer readable non-transitory medium storing instructions executable by a computer processor, the instructions when executed by the computer processor performing the steps comprising: (a) providing one or more reference signals; (b) forming a subspace representation of the reference signals to produce a learned manifold graph; (c) iteratively projecting successive noisy signals on the learned manifold graph; and (d) reconstructing the most likely shape of the successive input signal.

20. The computer readable non-transitory medium of any preceding embodiment, wherein the instructions when executed by the computer processor further perform steps comprising; extracting individual pulses from the plurality of reference signals; distilling at least one variable from the extracted pulses; normalizing the extracted pulses; and clustering similar normalized pulses to produce an idealized reference signal.

21. The computer readable non-transitory medium of any preceding embodiment, wherein the reconstructing the successive signal step comprises: estimating likely coordinates of successive samples in subspace with sequential tracking; and reconstructing the estimated coordinates back into input space using inverse mapping to produce a denoised waveform.

22. The computer readable non-transitory medium of any preceding embodiment, wherein the inverse mapping comprises: searching the k-nearest neighbors of a sample in the learned subspace; wherein the waveform estimate is effectively constrained and denoised.

23. The computer readable non-transitory medium of any preceding embodiment, wherein the instructions when executed by the computer processor further perform steps comprising: associating a label with measurable physiological conditions correlated with states of the reference biosignals; and labeling reconstructed reference signal states with at least one label from a library of labels.

Embodiments of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure(s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media, or can be stored remotely such as on a server or all or a portion of the instructions can be stored locally and remotely. Instructions stored remotely can be downloaded (pushed) to the device by user initiation, or automatically based on one or more factors.

It will further be appreciated that as used herein, that the terms processor, computer processor, central processing unit (CPU), and computer are used synonymously to denote a device capable of executing the instructions and communicating with input/output interfaces and/or peripheral devices, and that the terms processor, computer processor, CPU, and computer are intended to encompass single or multiple devices, single core and multicore devices, and variations thereof.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An apparatus for reducing noise in continuously monitored quasi-periodic biosignals without prior knowledge of the noise distribution, comprising:
(a) a computer processor; and
(b) a non-transitory computer-readable memory storing instructions executable by the computer processor;
(c) wherein said instructions, when executed by the computer processor, perform steps comprising:
(i) acquiring a plurality of reference signals;
(ii) forming a subspace representation of the reference signals to produce a learned manifold graph;
(iii) iteratively projecting successive signals on the learned manifold graph; and
(iv) reconstructing the most likely shape of the successive signal.

2. The apparatus of claim 1, wherein said instructions when executed by the computer processor further perform steps comprising:
extracting individual pulses from said plurality of reference signals;
distilling at least one variable from the extracted pulses;
normalizing the extracted pulses; and
clustering similar normalized pulses to produce an idealized reference signal.

3. The apparatus of claim 1, wherein said reference and successive signals are signals selected from the group consisting of electrocardiogram (ECG) signals, transcranial Doppler (TCD) signals, electroencephalogram (EEG) signals, and near infrared spectroscopy (NIRS) signals.

4. The apparatus of claim 1, wherein said instructions when executed by the computer processor further perform steps comprising:
acquiring a cerebral blood flow velocity (CBFV) waveform as a reference signal from a transcranial doppler (TCD) waveform, an ICP waveform, and an ICP elevation.

5. The apparatus of claim 1, wherein said subspace is obtained by a kernel discriminant analysis (KDA) of the reference signals solved using a spectral regression (SR) framework.

6. The apparatus of claim 1, wherein said reconstructing of the successive signal comprises:
estimating likely coordinates of successive samples in subspace with sequential tracking; and
reconstructing the estimated coordinates back into input space using inverse mapping to produce a denoised waveform.

7. The apparatus of claim 6, wherein said inverse mapping comprises:
searching k-nearest neighbors of a sample in subspace;
wherein the waveform estimate is effectively constrained and denoised.

8. The apparatus of claim 1, wherein said instructions when executed by the computer processor further perform steps comprising:
associating a label with measurable physiological conditions correlated with states of reference signals; and
labeling reference signal states with at least one label from a library of labels.

9. The apparatus of claim 8, wherein said library of labels comprises labels associated with cerebral blood flow velocity (CBFV) selected from the group consisting of degree of collateral blood flow circulation to a brain, quality of reperfusion after reperfusion therapy, lesion volume in acute stroke and traumatic brain injury, degree/presence of stenosis, presence of cerebral blood flow regulation dysfunction due to traumatic brain injury, presence of reperfusion injury, result of cerebral vascular reactivity (CVR) test, degree of success of intravascular treatment, and severity of vasospasms.

10. The apparatus of claim 1, wherein said instructions when executed by the computer processor further perform steps comprising:
assessing the quality of a signal by computing a difference between a denoised waveform and an original reference waveform;
wherein the larger the difference between signals, the lower the quality of the original signal.

11. A computer implemented method for reducing noise in continuously monitored quasi-periodic biosignals without prior knowledge of the noise distribution, the method comprising:
(a) acquiring one or more reference signals;
(b) forming a subspace representation of the reference signals to produce a learned manifold graph;
(c) iteratively projecting successive signals on the learned manifold graph; and
(d) reconstructing the most likely shape of a successive signal;
(e) wherein said method is performed by a computer processor executing instructions stored on a non-transitory computer-readable medium.

12. The method of claim 11, wherein said reference and successive signals are signals selected from the group consisting of electrocardiogram (ECG) signals, transcranial Doppler (TCD) signals, electroencephalogram (EEG) signals, and near infrared spectroscopy (NIRS) signals.

13. The method of claim 11, further comprising:
extracting individual pulses from said one or more reference signals;
distilling at least one variable from the extracted pulses;
normalizing the extracted pulses; and
clustering similar normalized pulses to produce an idealized reference signal.

14. The method of claim 11, wherein said subspace is obtained by a kernel discriminant analysis (KDA) of the reference signals solved using a spectral regression (SR) framework.

15. The method of claim 11, wherein said reconstructing of the successive signal comprises:
estimating likely coordinates of successive samples in subspace with sequential tracking; and
reconstructing estimated coordinates back into input space using inverse mapping to produce a denoised waveform.

16. The method of claim 15, wherein said inverse mapping comprises:
searching the k-nearest neighbors of a sample in subspace;
wherein a waveform estimate is effectively constrained and denoised.

17. The method of claim 11, further comprising:
associating a label with measurable physiological conditions correlated with states of each reference signal;
labeling reference signal states with at least one label from a library of labels; and
estimating a signal state in the learned manifold graph from the label during reconstruction.

18. The method of claim 11, further comprising:
assessing the quality of a signal by computing a difference between a denoised waveform and an original waveform;
wherein the larger the difference between waveforms, the lower the quality of the original signal.

19. A computer readable non-transitory medium storing instructions executable by a computer processor, said instructions when executed by the computer processor performing the steps comprising:
(a) acquiring a plurality of reference signals;
(b) forming a subspace representation of the reference signals to produce a learned manifold graph;
(c) iteratively projecting successive noisy signals on the learned manifold graph; and
(d) reconstructing the most likely shape of a successive input signal.

20. The computer readable non-transitory medium of claim 19, wherein said instructions when executed by the computer processor further perform steps comprising:
extracting individual pulses from said plurality of reference signals;
distilling at least one variable from the extracted pulses;
normalizing the extracted pulses; and
clustering similar normalized pulses to produce an idealized reference signal for forming the learned manifold graph.

21. The computer readable non-transitory medium of claim 19, wherein said reconstructing the successive signal step comprises:
estimating likely coordinates of successive samples in subspace with sequential tracking; and
reconstructing the estimated coordinates back into input space using inverse mapping to produce a denoised waveform.

22. The computer readable non-transitory medium of claim 21, wherein said inverse mapping comprises:
searching the k-nearest neighbors of a sample in the learned subspace;
wherein a waveform estimate is effectively constrained and denoised.

23. The computer readable non-transitory medium of claim 19, wherein said instructions when executed by the computer processor further perform steps comprising:
associating a label with measurable physiological conditions correlated with states of a reference signal; and
labeling reconstructed reference signal states with at least one label from a library of labels.
